(19) United States
(12) Patent Application Publication
(10) Pub. No.: US 2010/0120657 A1
(43) Pub. Date: May 13, 2010

(54) Reactive Polyorganosiloxanes Modified With Aromatic Substances

(75) Inventors: Horst Lange, Bochum (DE); Christopher Roos, Monheim (DE); Roland Wagner, Bonn (DE); Martin Kropfgans, Odenthal (DE)

Correspondence Address: RANKIN, HILL & CLARK LLP, 23755 Lorain Road - Suite 200, North Olmsted, OH, 44070-2224, US

(73) Assignee: MOMENTIVE PERFORMANCE MATERIALS GMBH, Leverkusen (DE)

(21) Appl. No.: 12/531,894

(22) PCT Filed: Mar. 19, 2008
(86) PCT No.: PCT/EP2008/053287

§ 371 Date: Oct. 23, 2009

(30) Foreign Application Priority Data
Mar. 19, 2007 (DE) 102007012909.4

(51) Int. Cl. C11D 3/20; C07F 7/10; A61K 8/58; A61K 8/35; A61K 8/33; B05D 1/02; B05D 1/18

(52) U.S. Cl. 510/466; 556/413; 512/21; 427/421.1; 427/430.1

(57) ABSTRACT

The invention relates to polyorganosiloxanes modified with fragrances and having reactive groups, to a method for coating a substrate using the polyorganosiloxanes according to the invention, use of the polyorganosiloxanes according to the invention for releasing the fragrance, to a method for producing the polyorganosiloxanes according to the invention as well as to their use, for example, in detergents or laundry detergents, in care products, in surface treatment agents and in cosmetics.

The invention relates to polyorganosiloxanes modified with fragrances and having reactive groups, to a method for coating a substrate using the polyorganosiloxanes according to the invention, use of the polyorganosiloxanes according to the invention for releasing the fragrance, to a method for producing the polyorganosiloxanes according to the invention as well as to their use, for example, in detergents or laundry detergents, in care products, in surface treatment agents and in cosmetics.

In many applications of cosmetic products and of everyday products for textile and home cleaning and care, there is a desire to obtain, during the application, a specific pleasant odor which lasts for as long as possible and is perceived as pleasant by the user.

In this regard, the use of reaction products of linear aminoalkylpolysiloxanes with ketone or aldehyde groups (EP 1062265 A1) was suggested. In case of contact with moisture at low pH values, the proposed products are subjected a hydrolytic cleavage, wherein the odiferous-substance molecule comprising keto groups or aldehyde groups and the aminoalkylpolysiloxane are being reformed. During use, however, the reformation of aminoalkylpolysiloxanes may give rise to bothersome secondary olfactory impressions, which, in particular, become stronger in proportion to the extent of evaporation of the released odiferous substance and to the effect of the aminoalkylpolysiloxane's own smell. Another problem of these compounds lies in the fact that, in liquid systems in particular, they are easily rinsed off the substrates, which also entails a loss of the fragrance capacity.

The present invention is therefore based on the object of providing fragrance-modified polyorganosiloxanes which on the one hand have high substantivity (i.e., durability of adhesion to a substrate) as well as on the other hand as little odor as possible after the release of the fragrance. This is an absolute requirement, in particular due to the high substantivity desired. Moreover, the improved substantivity makes it possible for the remaining polyorganosiloxane to permanently exhibit its useful properties, such as its surface-modifying properties, such as its softening properties, even after the fragrance has been released. Furthermore, the increase of substantivity of the polyorganosiloxanes according to the invention not only makes it possible to prolong the duration of fragrance release, but also permits a higher applied amount that leads to a more concentrated release of fragrance and thus, to a stronger scent. Moreover, this also permits using such fragrances whose scent is only perceivable at higher concentrations. Moreover, the low substantivity of the known fragrance-modified polyorganosiloxanes causes a relatively high release of the polyorganosiloxanes, in particular in liquid applications such as washing processes, which can lead to unwanted side effects during application, e.g. owing to the reduction of the desired foam formation in cleaner formulations, or to the unwanted formation of a silicone phase, which on the substrate causes troublesome silicone stains that have a fatty appearance and can be removed only with great difficulty. Another advantage of the fragrance-modified polyorganosiloxanes according to the invention comprising reactive groups lies in the fact that, if required, other active molecules, such as other fragrances in the sense of a fragrance bouquet, optionally also the fragrances with different release kinetics, can be introduced into the polyorganosiloxane via the reactive groups. In particular, different fragrances can be introduced in this way in order thus to obtain a characteristic scent and to control and change the perceived character of the scent after the application over time. This is not possible in the prior art. Moreover, only polyorganosiloxanes with a linear structure without reactive groups are being described there, so that, owing to their structure, they do not possess a particularly pronounced affinity to the surfaces to be treated, and exhibit neither favorable efficiency as regards deposition on a treated substrate surface, nor good persistence on the treated surface. Because of its high substantivity, the fragrance-free organopolysiloxane obtained from the polyorganosiloxane according to the invention after the fragrance has been cleaved off displays its caring or conditioning effect on the substrates without becoming conspicuous due to an unpleasant smell.

Accordingly, this presented the object of finding a suitable alternative to the prior art that is both technically and economically attractive and to find a simple method for producing these compounds which avoids the above-described drawbacks of the prior art.

This object was achieved by providing fragrance-modified polyorganosiloxanes having reactive groups.

The aforementioned reactive groups are, in particular, such groups which are capable of reacting with a substrate and/or of condensation.

Substrates that are capable of reacting with the polyorganosiloxanes according to the invention include the following, for example: fibers, textiles, plasters, paper, wallpapers, molded articles, ceramics, paint layers, foils, hair, skin, wood, glass, plastics, etc.

The reactive groups in the polyorganosiloxanes according to the invention particularly include those of Formula (I):

—SiR²ₙX₃₋ₙ (I),

wherein
n=0 to 2,
R² is selected from optionally substituted alkyl and phenyl,
X is selected from halogen, —OR³, —OC(O)R³, —N═CR³₂, —NR³₂, —NC(O)R³, —R⁴—Y,
wherein R³ is selected from the group consisting of hydrogen, optionally substituted alkyl, optionally substituted alkenyl, optionally substituted cycloalkyl and aryl,
R⁴ is a divalent optionally substituted organic residue optionally comprising one or more heteroatoms, and
Y is selected from the group consisting of —NCO, —OH, —NR³₂, —C(O)Cl, —SO₂Cl, —SO₂-vinyl (vinyl sulfone), triazinyl, halogen triazinyl, pyrimidinyl.

Functional groups on the substrate that are capable of reacting with the above reactive groups of the polyorganosiloxanes according to the invention are located, in particular, at the surface of the substrates, and are selected, for example, from the group consisting of: hydroxy, amino, carboxy, carbonyloxy (ester), disulfide, mercapto, etc.

The polyorganosiloxanes according to the invention can be obtained, in particular, by reacting polyorganosiloxanes comprising functionalized, reactive groups with fragrances, or by condensation of fragrance-modified polyorganosiloxanes or organosilanes.

Fragrances contained, according to the invention, in the polyorganosiloxanes preferably comprise at least one functional group selected from the group consisting of keto, aldehyde and/or hydroxy groups, which react with suitable functional groups of the polyorganosiloxanes or organosilanes used as starting materials. Accordingly, the functionalized polyorganosiloxanes or organosilanes used as starting compounds comprise at least one functional group capable of reacting with keto-, aldehyde- and/or hydroxy-functionalized fragrances, wherein, according to the invention, at least one reactive group must remain in the obtained polyorganosiloxane after the reaction with the fragrance. Said functional groups on the polyorganosiloxanes or organosilanes that react with the fragrance are preferably selected from the group consisting of aminoalkyl groups, hydroxyalkyl groups and alkoxy groups respectively bonded to a silicon atom. It must be remarked in this context that the above-mentioned functional groups may also be the reactive groups remaining on the polyorganosiloxane. The functional group may thus be an alkoxysilyl group, for example, which reacts with a fragrant alcohol only partially, so that alkoxysilyl groups remain in the resulting polyorganosiloxane of the invention and are thus available for the reaction with a substrate or for the condensation of the polyorganosiloxanes (after the admission of water). In a preferred embodiment of the invention, the functionalized polyorganosiloxanes or organosilanes used as starting compounds comprise at least one primary or secondary amine residue.

The fragrance-modified polyorganosiloxanes within the meaning of the invention in particular comprise the following silicon structural units:

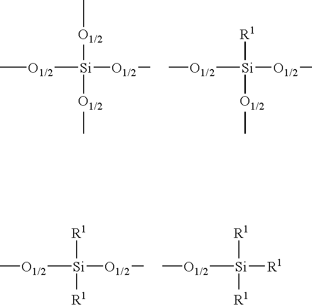

wherein
R¹ is selected from the group consisting of:
   optionally substituted, straight-chain, branched or cyclic saturated or unsaturated, alkyl, optionally substituted aryl, in particular methyl, ethyl, vinyl, allyl, propyl, octyl, dodecyl, capryl, stearyl, phenyl, phenylethyl, phenylpropyl, limonenyl, cyclohexylethyl, norbornenyl, optionally interrupted by one or more heteroatoms,
   fragrance-releasing groups capable of cleaving off one or more fragrance molecules,
   reactive groups capable of reacting with substrates,
      wherein the fragrance-modified polyorganosiloxanes contain on average at least one fragrance-releasing group per molecule.

Expediently, the polyorganosiloxanes according to the invention contain at least one structural element of the formula:

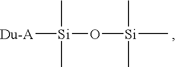

wherein
the group ‘Du’ represents a fragrance-derived structural unit from which the fragrance is released again by cleaving,
A represents a silicon-fee spacer unit, and
the free valences on the silicon atoms are saturated by residues selected from organic residues and siloxane residues, provided that at least one of the free valences is saturated by a siloxane residue.

Expediently, the polyorganosiloxanes according to the invention contain on average at least two, preferably at least 5, still more preferably 8, preferably not more than 1000, more preferably not more than 200, still more preferably not more than 50 siloxy groups per molecule.

Fragrance-containing structural units are in particular derived from fragrances containing keto, aldehyde and/or hydroxy groups.

Suitable ketones, aldehydes or alcohols traditionally used in perfume production are for example those mentioned in “Perfume and Flavor Chemicals”, Volume I and II, S. Arctander, Allured Publishing, 1994, ISBN 0-931 71 0-35-5.

Fragrant ketones include, for example: buccoxime; iso-jasmone; methyl beta-naphthyl ketone; musk indanone; tona-lid/musk plus; alpha-damascone, beta-damascone, delta-damascone, iso-damascone, damascenone, damarose, methyl dihydrojasmonate, menthone, carvone, camphor, fenchone, alpha-ionone, beta-ionone, gamma-methyl known as ionone, fleuramone, dihydrojasmone, cis-jasmone, iso-E-Super□, methylcedrenylketone or methylcedrylone, acetophenone, methyl acetophenone, para-methoxyacetophenone, methyl beta-naphthyl ketone, benzyl acetone, benzophenone, para-hydroxyphenylbutanone, celery ketone or livescone, 6-isopropyldecahydro-2-naphtone, dimethyloctenone, frescomenthe, 4-(1-ethoxy-vinyl)-3,3,5,5-tetramethylcyclohexanone, methylheptenone, 2-(2-(4-methyl-3-cyclohexene-1-yl)propyl)cyclopentanone, 1-(p-menthene-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)indanone, 4-damascol, dulcinyl or cassione, gelsone, hexalon, isocyclemone E, methyl cyclocitrone, methyl-lavender-ketone, orivon, para-tertiary-butyl-cyclohexanone, verdone, delphone, muscone, neobutenone, plicatone, veloutone, 2,4,4,7-tetramethyl-oct-6-en-3-one, tetrameran.

The perfume ketones are preferably selected from alpha-damascone, delta-damascone, iso-damascone, carvone, gamma-methylionone, Iso-E-Super® (7-acetyl-1,2,3,4,5,6,7,8-octahydro-1,1,6,7-tetramethyl naphthalene), 2,4,4,7-tetramethyl-oct-6-en-3-one, benzyl acetone, beta-damascone, damascenone, methyl dihydrojasmonate, methyl cedrylone and mixtures thereof.

Alpha-damascone is particularly preferred.

Fragrant aldehydes include, for example: adoxal; anisic aldehyde; cymal; ethyl vanillin; florhydral; helional; heliotropin; hydroxycitronellal; koavone; lauric aldehyde; lyral; methyl nonyl acetaldehyde; p-t-bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-(para-methoxyphenyl)propanal, 2-methyl-4-(2,6,6-trimethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal; decyl aldehyde, 2,6-dimethyl-5-heptenal, 4-(tricyclo[5.2.1.0(2.6)]-decylidene-8)-butanal, octahydro-4,7-methano-1H-indenecarboxaldehyde, 3-ethoxy-4-hydroxybenzaldehyde, para-ethyl-alpha,alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, undecenal, 2,4,6-trimethyl-3-cyclohexene-1-carboxaldehyde, 4-(3) (4-methyl-3-pentenyl)-3-cyclohexene-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl) propanal, dihydrocinnamic aldehyde, 1-methyl-4-(4-methyl-3-pentenyl)-3-cyclohexene-1-carboxaldehyde, 5- or 6-methoxyhexahydro-4,7-methanoindan-1 or 2- carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, para-tolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl) benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, hexanal, trans-2-hexanal, 1-p-menthene-q-carboxaldehyde and mixtures thereof.

Preferred aldehydes are selected from: 1-decanal, benzaldehyde, florhydral, 2,4-dimethyl-3-cyclohexene-1-carboxaldehyde; cis/trans-3,7-dimethyl-2,6-octadien-1 -al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, p-t-bucinal, lyral, cymal, methyl nonyl acetaldehyde, hexanal, trans-2-hexenal, and mixtures thereof.

Preferred fragrant alcohols are selected, for example, from: 2-methylbutanol, 3-pentanol, n-pentanol, 2-pentanol, n-hexanol, 2-methylpentanol, 1-decanol, sandela, nonadol, dimetol, thymol, 1-heptanol, menthol, eugenol, vanillin, o-vanillin, 4-(p-hydroxyphenyl)-2-butanone, syringe aldehyde, prenol, cis-3-hexanol, trans-3-hexanol, cis-4-heptenol, trans-2-octenol, trans-2-cis-6-nonadienol, geraniol, nerol, ebanol, citronellol, crotyl alcohol, oleyl alcohol, linalool, α-terpineol, β-phenethyl alcohol, cinnamic alcohol, benzyl alcohol, α-methylbenzyl alcohol, nonyl alcohol, 1-octanol, 3-octanol, phenethyl salicylate, hydrocinnamyl alcohol, cis-6-nonen-1-ol, trans-2-nonen-1-ol, methyl salicylate, cis-3-octen-ol, anisyl alcohol, carvacrol, dihydrocarveol, benzyl salicylate, tetrahydrogeraniol, ethyl salicylate, ethyl vanillin, isoeugenol, isopulegol, lauryl alcohol, tetrahydrolinalool, 2-phenoxyethanol, citronellol, eugenol, farnesol, thymol and geraniol. Compounds of this kind are described, for example, in EP 0 799 885, EP 0 771 785, WO 96/38528, U.S. Pat. No. 5,958,870.

Silicon-free spacer units are in particular selected from divalent, saturated hydrocarbon residues comprising one or more heteroatoms, with up to 30 carbon atoms.

In a preferred embodiment of the invention, one or more methyl groups in the methyl siloxane compounds are replaced by phenyl groups in order to obtain resinous compounds melting at, for example, 40 to 120° C., in order on the one hand to be able to store the compound in the solid form and, on the other hand, to attain liquefaction of the compound, for example during a washing process, and thus, better dispersibility.

In a particularly preferred embodiment of the invention, the solids are gel-like, and the branched fragrance-modified polyorganosiloxanes have a penetrometer value (DIN ISO 2137, 2nd edition—1985-11-01) at 25° C. of less than 50 mm/6 sec with the cone C2 (62.5 g) in the beaker B2.

The term solids also includes elastomer solids which according to DIN 53505 must have a hardness according to Shore A of more than 10° if a molded plate, a molten body or a molding with a thickness of 6 mm is tested at 25° C.

This means that, in a preferred embodiment, the fragrance-modified polyorganosiloxanes according to the invention are viscous to non-flowable, solid, in particular solid masses at room temperature (25° C.). Apart from the low odor intensity of the functionalized branched polysiloxane remaining after the fragrance has been cleaved off, which is due to its low vapor pressure, one advantage of this embodiment is the possibility of incorporating the non-flowable, in particular solid masses into solid formulations, such as paste-like or in particular powdery formulations such as washing powders, optionally after disintegration of the masses. In this way, it may in particular cease to be necessary that the polyorganosiloxanes are applied onto carries, and thus, relatively high fragrance concentrations can be obtained in relation to the weight or volume, so that the polyorganosiloxanes provide a high odorous capacity.

By using phenyl siloxy groups, in particular by using the structural unit PhSiO_{3/2} in the polyorganosiloxanes according to the invention, compounds preferably meltable at 40 to 120° C. are obtained, with the above-mentioned advantages. Therefore, in a preferred embodiment of the invention, the polyorganosiloxanes preferably comprise at least one phenylsiloxy group, preferably at least one PhSiO_{3/2} unit.

Melting point or range means that, in a differential thermo calorimeter (DSC), the melting temperature is determined by measuring a melting enthalpy of >0 joule at this temperature if the sample is heated from a lower temperature at a rate of 0.5 to 5 K/min.

In a preferred embodiment, reactive siloxane structures are selected which bond to the substrate, preferably with the fiber of textiles in such a way that the reaction products of the silioxane fragrance and the fiber cause no discoloration on the substrates below 180° C., more preferably below 250° C. Discoloration means that white pieces of fabric, after having been subjected to temperatures of up to 180° C., more preferably to 250° C., for 5 minutes, do not exhibit any discoloration greater than that of an untreated fabric after this treatment, so that they withstand even extreme heating under an iron for 5 minutes without disadvantageous discoloration.

The polyorganosiloxanes, preferably polydimethylsiloxanes, used as starting compounds comprise at least one functional group capable of reacting with keto-, aldehyde- and/or hydroxy-functionalized fragrances. This functional group includes in particular groups that are selected from aminoalkyl groups, secondary aminoalkyl groups, such as alkylaminoalkyl groups, cycloalkyl groups, such as cyclohexyl aminoalkyl groups, or arylaminoalkyl groups, hydroxyalkyl groups and alkoxy groups, respectively bonded to one silicon atom. According to the invention, these groups also include polyaminoalkyl and polyhydroxyalkyl groups that are suitable for releasing more than one fragrance molecule per residue R¹.

Preferred Si-bonded aminoalkyl groups include in particular the aminopropyl and the aminomethyl group. Analogously, preferred hydroxyalkyl groups include the hydroxypropyl and the hydroxymethyl groups.

The starting compounds having such functional groups may for example be the following compounds:

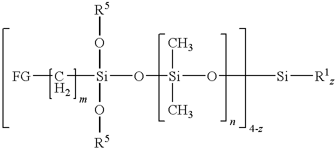

wherein FG is the functional group capable of reacting with the fragrance, R¹ is defined as above,
R⁵ is a C₁ to C₁₀ alkyl, C₆-C₁₀ cycloalkyl or aryl residue,
m=1 to 8, and
z=0, 1, 2 or 3,

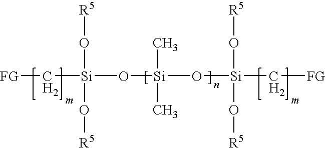

wherein R⁵, FG, n and m are defined as indicated above, such as, in particular, compounds of the formula:

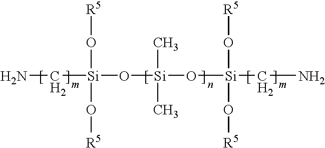

wherein R⁵, m and n are defined as indicated above,
such as, for example H₂NCH₂CH₂CH₂—Si(OEt)₂—[SiMe₂O]₁₀-OSi(OEt)₂—CH₂CH₂CH₂NH₂.

By reacting the above-mentioned functional group capable of reacting with the fragrance, the following fragrance-releasing groups -A-Du are formed, for example:

For example, the following group -A-Du with ‘D’=fragrance molecule without bonding group is formed from a aminoalkyl residue at the silicon of a siloxane residue and a fragrance containing keto and/or aldehyde, i.e., subtituents or structural units for completing the molecular structure of a fragrance are in each case:

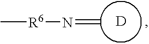

wherein
R⁶ is a C₁ to C₈ alkylene residue, and

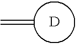

is a residue which formally emerges from oxygen cleaving off from an O═C group of a fragrance.

A competitive addition may occur in the case of α,β-unsaturated carbonyl fragrances: for example, the aminoalkyl residue in the siloxane may be added to a α,β-unsaturated carbonyl fragrance, wherein the following residue corresponding to -A-Du is formed:

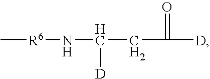

wherein R⁶ is defined as indicated above, and the residue

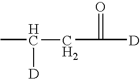

from the α,β-unsaturated carbonyl fragrance

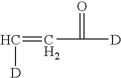

results from the addition of the aminoalkyl residue to the C═C double bond of the α,β-unsaturated carbonyl fragrance, with D in each case being substituents for completing the molecular structure of a fragrance.

Moreover, a hemiacetal or hemiketal, for example, may form as the group -A-Du from a hydroxyalkyl residue at the siloxane and a fragrance containing keto and/or aldehyde:

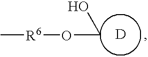

wherein R⁶, is defined as indicated above, and

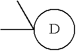

is a residue which formally emerges from oxygen cleaving off from an O═C group of a fragrance, is de facto formed naturally from the reaction of a hydroxyalkyl group with the keto group of the fragrance

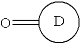

Analogously, two hydroxyalkyl residues at the siloxane and a fragrance containing keto and/or aldehyde can form an acetal or ketal:

-A-Du in that case formally corresponds to a residue -(A)₂-Du having the formula:

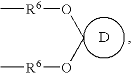

wherein
R⁶ is defined as indicated above, and the residue

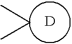

is a residue which formally emerges from oxygen cleaving off from an O═C group of a fragrance

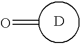

and is de facto formed naturally from the reaction of two hydroxyalkyl groups at the silicon with a keto or aldehyde group of the fragrance.

Moreover, the following fragrance-generating groups can be formed from alkoxy groups bonded to the silicon and hydroxy-containing fragrances:

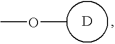

wherein

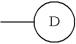

formally emerges from the hydroxy group cleaving off from a hydroxy-containing fragrance and emerges de facto from the reaction.

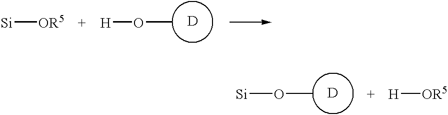

This reaction also includes the possibility of a reaction of a fragrance containing a keto and/or aldehyde in its enol-form.

Examples of fragrances containing keto and/or aldehyde also include mixtures thereof.

The concentration of the fragrance-releasing groups in the polyorganosiloxanes according to the invention relative to the number of siloxy units preferably is at least approximately 1 mole-% to 200 mole-%. A concentration exceeding 100 mole-% is made possible by the appropriate use of -A-Du-polysubstituted siloxy groups. Preferred concentrations are approximately 10 to 100 mole-%. Particularly preferably, the appropriate concentration is 12 to 80 mole-%.

In addition to the fragrance-releasing groups -A-Du, the fragrance-modified polyorganosiloxanes according to the invention comprise groups capable of reaction with the substrates. As was already explained in the introduction, this has numerous advantages. On the one hand, it causes an immobilization of the polyorganosiloxane on a substrate on which the polyorganosiloxane displays its advantageous effects, particularly on the surface of the substrate, which, apart from the release of the fragrance, include in particular the softening, hydrophobing and hydrophilizing effects. Moreover, the fragrance-modified polyorganosiloxane, after having been immobilized on the substrate, can release the fragrance in a delayed manner, i.e. in particular over a long period of time. Furthermore, immobilization using the groups mentioned which are reactive with regard to the substrate leads to the polyorganosiloxane not generating any odor anymore after the fragrance has been released, because of its immobilization. Preferred substrates to which the polyorganosiloxanes, which were provided with groups that are reactive with regard to the substrate, can react include in particular fibers, plasters, wallpapers, molded plastic articles, ceramics, paint layers, foils, hair, skin and wood. Preferred groups in the polyorganosiloxane capable of reacting with the substrate include reactive functional groups having the formula (I):

—SiR²ₙX₃₋ₙ (I),

wherein
n=0 to 2,
R² is selected from optionally substituted alkyl and phenyl,
X is selected from halogen, —OR³, —OC(O)R³, —N═CR³₂, —NR³₂, —NC(O)R³, —R⁴—Y,
wherein R³ is selected from the group consisting of hydrogen, optionally substituted alkyl, optionally substituted alkenyl, optionally substituted cycloalkyl and aryl,
R⁴ is a divalent optionally substituted organic residue optionally comprising one or more heteroatoms, and
Y is selected from the group consisting of —NCO, —OH, —NR³₂, —C(O)Cl, —SO₂Cl, —SO₂-vinyl (vinyl sulfone), triazinyl, halogen triazinyl, pyrimidinyl.

The preferred reactive group capable of reacting with a substrate is the alkoxy group.

The concentration of the reactive groups in the polyorganosiloxanes according to the invention relative to the number of siloxy units preferably is at least approximately 1 mole-% to 200 mole-%. A concentration exceeding 100 mole-% is made possible by the appropriate use of polysubstituted siloxy groups. Preferred concentrations are approximately 10 to 100 mole-%. Particularly preferably, the appropriate concentration is 20 to 80 mole-%.

The case in which the groups capable of reacting with the substrate can also serve for the condensation of the polyorganosiloxanes amongst each other is also included according to the invention. This particularly includes the case in which a part of the reactive groups reacts in the condensation of the polyorganosiloxane and another part of the reactive groups reacts with the substrate. The alkoxy silyl group, in particular, is a reactive functional group that is capable of fulfilling the aforementioned dual function.

The substrates to which the polyorganosiloxanes according to the invention are preferably bonded preferably have on their surface functional groups that are selected, for example, from the group consisting of: hydroxy, amino, carboxy, carbonyloxy (ester), disulfide, mercapto. It is particularly preferred that the substrates comprise hydroxy groups.

The starting compounds preferably used in the production of the polyorganosiloxanes are polyorganosiloxanes which preferably comprise at least one primary or secondary amine residue.

In another embodiment of the invention, the polyorganosiloxanes can be immobilized on a carrier. Suitable carriers include, for example, silicic acids, zeolites, cyclodextrins, kaolins, bentonites, polyalkylene oxide waxes, polyacrylates, etc. In the process, the bonding of the polyorganosiloxanes takes place, in particular, in the same manner as in the case of the above-mentioned substrates. That is, reactive groups of the polyorganosiloxanes, such as alkoxysilyl compounds, can react with the carrier, which may for example include hydroxy groups on the surface of the carrier.

Naturally, the polyorganosiloxanes according to the invention are used in particular as fragrances, more specifically as fragrance-releasing so-called “profragrances”, that is, used for releasing the fragrance.

In one embodiment of the invention, this in particular relates to a method for producing a scent which comprises the treatment of the substrate, which was already mentioned above, with at least one polyorganosiloxane according to the invention, and the release of the fragrance therefrom.

The invention furthermore relates to a method for producing the polyorganosiloxanes according to the invention comprising the reaction of functionalized polyorganosiloxanes with the fragrances, as was already outlined above. Alternatively, the polyorganosiloxanes according to the invention may also be produced by an optionally catalyzed condensation with an admission of water, in particular of alkoxy silyl groups of fragrance-modified polyorganosiloxanes or organosilanes. Suitable fragrance-modified organosilanes are described, for example, in WO 2005/041908. The fragrance-modified organosilanes of WO 2005/041908 are in particular of the type DU-A-Si(OR)₃, which in this form are completely unsuitable for achieving the object according to the invention because their vapor pressure is too high, so that they can lead to an impairment of the odor after the fragrance has been cleaved off.

In one embodiment of the invention, the polyorganosiloxane obtained is applied onto a carrier material in particular by spray drying or fluid bed granulation. The supported material thus produced can contain the polyorganosiloxane because of adsorption or bonded to the carrier material by a chemical reaction.

In the case of the production method which includes the reaction of functionalized polyorganosiloxanes or organosilanes comprising condensable groups capable of forming Si—O—Si-bonds with fragrances and subsequent condensation while forming branched polyorganosiloxanes, it is found, surprisingly, that this reaction can be carried out such that the alkoxypolysiloxane components, which are in fact hydrolytically sensitive, are completely or partially preserved, despite the condensation of, for example, amino alkyl groups with, for example, the ketonic or aldehydic fragrances, which may proceed with dehydration, so that they are still available for condensation while forming branched polyorganosiloxanes or for reaction with a suitable substrate.

The polyorganosiloxanes according to the invention advantageously can be produced both by discontinuous as well as continuous reaction of appropriate alkoxy aminoalkylpolysiloxanes with ketonic and aldehydic odiferous substances. For this purpose, mixtures of the alkoxy aminoalkylpolysiloxanes are preferably used, in particular with the ketonic or aldehydic fragrances, which are heated to temperatures of more than 40° C., preferably of more than 100° C. The reaction can be carried out such that a solvent which, together with the water formed in the condensation reaction, forms an azeotrope that can be separated by destillation, is added to the reaction mixture. If the alkoxy aminopolysiloxanes are used, the use of azeotrope-forming entrainers for separating water can be dispensed with. This practice is particularly advantageous in that both the bonding of the fragrance as well as the condensation can be carried out in a single reaction stage, with the released water causing the condensation of the alkoxy silyl groups. Another advantage of this practice lies in the fact that, because the water does not have to be removed, higher temperatures are avoided, which permits an economical production of very pure products and makes complex separation steps for removing undesired residual entrainer contaminations or decomposition products dispensable. By controlling the ratio of alkoxy silyl groups to the amount of water released in the reaction system and/or the amount of water added to the reaction system, the amount of alkoxy groups remaining in the final product can be controlled, and thus the reactivity with regard to certain substrates can be maintained or controlled.

The alkoxy groups of the polyorganosiloxanes or organosilanes can be condensated even after the reaction with the fragrance following the addition of water by using suitable condensation catalysts, such as, in particular, organometallic compounds, such as organotin compounds, such as dibutyltinlaurate, organotin oxides, organometallic compounds, such as carboxylates, alcoholates or chelates of titanium, calcium, aluminum, zirconium or zinc. In another preferred embodiment, fragrant alcohols can additionally be added to the reaction mixture of alkoxy aminoalkylpolysiloxane and the ketonic or aldehydic fragrance prior to, during or after the amine-ketone or the amine-aldehyde reaction, respectively, the fragrant alcohols being capable of interesterification reactions with the alkoxy silyl groups.

In another embodiment of the invention, different fragrances, for example, with, for example, different chemical bonds to the polysiloxane polymer skeleton are obtained. In the case of contact with moisture, the chemically bonded odiferous substances are released from such substances with different reaction rates. Accordingly, this embodiment offers the possibility of changing the characteristics of the fragrance during the release time using different chemical bonds and/or chemically different fragrances.

The polyorganosiloxanes according to the invention also exhibit, in particular, good separation efficiency from an application solution, such as an emulsion or microemulsion, and their high surface affinity can be exploited to achieve surface-caring or conditioning effects, such as softening, fiberelastic effects, or color-preserving, color-enhancing or glossy effects.

The fragrance-modified polyorganosiloxanes according to the invention are used in the following functions or applications, for example, in which they serve as fragrance donors or for imparting further properties, such as softening properties.

In detergents, such as laundry detergents, washing-up detergents, in care products, such as textile care products, fragrant strips based on paper or textile materials, fragrance donors in soaps or soap formulations, WC fragrance donors, in wallpapers, in paper, as impregnating agents in sanitary facilities, in fragrant inlay soles, in clothes care products, in sanitary pads, as textile care products prior to, during and after washing, in particular in “rinse-off” applications, in surface treatment products, such as in floor polishers, in cosmetics, such as deodorizing agents, make up, such as mascara, skin care products, hair cosmetics, such as shampoos, hair care products, hair gels, styling gels.

Particularly preferably, the polyorganosiloxanes according to the invention are used in compositions, in particular detergents, such as laundry detergents, washing-up liquids, as described in the European Patents 1095128, 1123376, 1161515, 1062265, 1144578, 1144579, 1360269, 1661978, 1280882, 1383858, WO 2005-105970 and in WO 2006-029188. As a rule, such detergents comprise non-ionic, anionic and/or cationic surfactants.

For this purpose, the fragrance-modified polyorganosiloxanes comprising reactive groups, in particular in the case where they are used in aqueous compositions, are first applied to a carrier or dispersed or encapsulated in a solid or liquid non-miscible carrier. In the case where they are used in a non-aqueous composition, the fragrance-modified polyorganosiloxanes can be mixed directly into a perfume oil composition or mixed into the non-aqueous carrier or solvent.

Moreover, the invention provides a composition of the fragrance-modified polyorganosiloxanes according to the invention containing at least one inorganic or organic acid. It was found that the fragrance-modified branched polyorganosiloxanes according to the invention can be stabilized in such composition, and that such compositions are therefore suitable in particular for producing stabilized concentrates, so-called perfume oils. Naturally, these forms are suitable in particular as a sales form of the fragrance-modified branched polyorganosiloxanes according to the invention.

Examples of suitable acids include, for example, carboxylic acids, preferably hydroxycarboxylic acid, and citric acid is much preferred. Examples of inorganic acids include hydrochloric acid, phosphoric acid, sulfuric acid, nitric acid.

Besides the fragrance-modified branched polyorganosiloxanes according to the invention, the perfume oils can contain other fragrances, solvents, such as alcohols, esters, ketones.

Fragrance-modified branched polyorganosiloxanes according to the invention:
100 parts by wt. fragrance-modified polyorganosiloxane
0 to 100 parts by wt. solvent, preferably C₁-C₅ alkyl alcohol, more preferably without solvent, with addition of heat.
0.5-1 mole acid per basic equivalent nitrogen, with citric acid, formic acid or HCl being preferred.

The amine content is determined by titration as a change in color from tetrabromophenolphthaleine in isopropanol/xylol 1:1 by acid-base titration.

Because of the special properties, the fragrance-modified branched polyorganosiloxanes according to the invention are capable of permanently attaching to the substrates treated with them, to modify the surface properties of the substrates in a beneficial way in the process and to simultaneously fixate the fragrance on the substrate, from which it is released over a long period of time.

Accordingly, the invention furthermore relates to detergents, care products, surface treatment agents and cosmetic agents containing at least one fragrance-modified polyorganosiloxane of the invention.

The fragrance-modified branched polyorganosiloxanes according to the invention can preferably be used in compositions such as laundry detergent compositions, detergent compositions, in particular for hard surfaces, and compositions for body hygiene, wherein the fragrance-modified branched polyorganosiloxanes are mixed with one or more laundry detergent or detergent components.

Incorporation of the fragrance-modified branched polyorganosiloxanes according to the invention can expediently take place by incorporation, for example by spraying or addition in dry form. According to the invention, the fragrance-modified branched polyorganosiloxanes can for example be incorporated into laundry detergent compositions, including those in liquid or solid form, such as powder and tablets, and in softening compositions including softening compositions added to the rinsing prior to, during or after the washing or cleaning process, and softening compositions added to the drier. With respect to common softener compositions into which the fragrance-modified branched polyorganosiloxanes according to the invention can be incorporated, reference can be made, for example, to EP-A-971 025, the entire content of disclosure of which is incorporated herein by reference. Preferably, the fragrance-modified branched polyorganosiloxanes according to the invention are added to a laundry detergent composition, preferably in a solid form. Finished compositions usually contain the fragrance-modified branched polyorganosiloxanes according to the invention in an amount of 0.1 to 25% by wt., more preferably 0.2 to 10% by wt., and most preferably 0.5 to 5% by wt. Laundry detergent compositions containing the fragrance-modified branched polyorganosiloxanes according to the invention preferably have a bleaching agent precursor, a source for alkaline hydrogen peroxide required for forming a peroxy acid bleaching agent in the washing solution, and preferably contain also other constituents commonly used for laundry detergent compositions. These include, for example, one or more surfactants, organic and inorganic builders, dirt-suspending and anti-resettling agents, anti-foaming agents (antifoam), enzymes, fluorescent whiteners, photoactive bleaching agents, fragrances, colorants, clay softeners, effervescent agents and mixtures thereof. Typical constituents can be found in EP-A-0 659 876 and EP-A-971 024, the content of disclosure of which is completely included in the present patent application. The laundry detergent compositions containing the fragrance-modified branched polyorganosiloxanes according to the invention can preferably contain clay, which is present in a concentration of 0.05% by wt. to 40% by wt., more preferably of 0.5% by wt. to 30% by wt, most preferably of 2% by wt. to 20% by wt. in the composition. A preferred clay can be bentonite clay. Smectite clays, such as disclosed in U.S. Pat. Nos. 3,862,058, 3,948,790, 3,954,632 and 4,062,647 and in the European patents Nos. EP-A-299 575 and EP-A-313 146 are much preferred. Specific examples of suitable smectite clays include those selected from the classes of the montmorillonites, hectorites, volkonskoites, nontronites, saponites and sauconites, particularly those that possess an alkaline or alkaline earth metal ion within the crystalline lattice structure. Sodium or calcium montmorillonite are particularly preferred. Clays have for example an average particle size of 10 nm to 800 nm, more preferably of 20 nm to 500 nm, most preferably of 50 nm to 200 nm. The smectite clays suitable in this context typically have a cation exchange capacity of at least 50 meq/100 g. The crystalline lattice structure of the clay mineral compounds, in a preferred embodiment, can contain a substituted cationic fabric softener. Such substituted clays are called “hydrophobically activated” clays. The cationic fabric softeners are typically present in a weight ratio of cationic fabric softener to clay of 1:200 to 1:10, preferably of 1:100 to 1:20. Suitable cationic fabric softeners include the water-insoluble tertiary amines or the double long-chain amide materials as disclosed in GB-A-1 514 276 and EP-B-0 011 340. A preferred commercially available, “hydrophobically activated” clay is a bentonite clay containing approximately 40% by wt. of quaternary dimethyl ditallow ammonium salt (Claytone EM). The clay can be present in an intimate mixture or in a particle such as a humectant and a hydrophobic compound, preferably a wax or an oil, such as paraffin oil. Organic compounds, including propylene glycol, ethylene glycol, dimers or trimers of glycol, most preferred glycerin, are preferred humectants. The particle preferably is an agglomerate. As an alternative, the particle may be constituted such that the wax or the oil and optionally the humectant form a covering for the clay, or, as an alternative, the clay can be a covering for the wax or the oil and the humectant. It may be preferred that the particle includes an organic salt or silicon dioxide or silicate. However, the clay is preferably mixed with one or more surfactants and optionally builders and optionally water, with the mixture preferably being dried thereafter. Preferably, such a mixture is processed further in a spray drying process in order to obtain a spray-dried particle comprising the clay. It may also be preferred that the flocculant is also incorporated into the particle or the granule comprising the clay. It may also be preferred that the intimate mixture comprises a masking agent. The compositions of the invention can contain a clay flocculant, which preferably is present in a concentration of 0.005% by wt. to 10% by wt., more preferably of 0.05% by wt. to 5% by wt, most preferably of 0.1% by wt. to 2% by wt. of the composition. The clay flocculant fulfils the function of bringing together the particles of the clay compound in the washing solution and thus supporting their deposition on the surface of the fabric during washing. Preferred clay flocculants in this case include organic polymer materials with an average weight of 100,000 to 10,000,000, preferably of 150,000 to 5,000,000, more preferably of 200,000 to 2,000,000. Suitable organic polymer materials include homopolymers or copolymers containing monomer units selected from alkylene oxide, in particular ethylene oxide, acrylamide, acrylic acid, vinyl alcohol, vinyl pyrrolidone and ethyleneimine. Homopolymers, in particular of ethylene oxide, but also of acrylamide and acrylic acid, are preferred. The European patents Nos. EP-A-299 575 and EP-A-313 146 describe preferred organic polymer clay flocculants for use in this context. The weight ratio of clay to the flocculant polymer is preferably 1000:1 to 1:1. In this case, inorganic clay flocculants ar also suitable; typical examples thereof include lime and alum. The flocculant preferably is present in a laundry detergent base grain, such as a laundry detergent agglomerate, extrudate or spray-dried particle, which generally comprises one or more surfactants and builders. Effervescent agents may optionally also be used in the compositions of the invention. Examples for acid and carbonate sources and other effervescent systems can be found in Pharmaceutical Dosage Forms: Tablets, Volume 1, pages 287 to 291. Suitable inorganic alkaline and/or alkaline earth carbonate salts include carbonate and hydrogen carbonate of potassium, lithium, sodium etc., of which sodium and potassium carbonate are preferred. Suitable bicarbonates for use in this context include all alkaline metal salts of bicarbonate, such as lithium, sodium, potassium etc., of which sodium and potassium bicarbonate are preferred. However, the choice between carbonate or bicarbonate or mixtures thereof can be made dependent upon the desired pH value in the aqueous medium in which the granules are dissolved. Other preferred optional constituents comprise enzyme stabilizers, polymer dirt repellents, substances inhibiting the transfer of dyes from one fabric to another during the cleaning process (i.e. dye transfer inhibitors), polymeric dispersing agents, anti-foaming agents, optical brightening agents or other brightening agents or whiteners, antistatic agents, other active constituents, carriers, hydrotropic substances, processing aids, dyes or pigments, solvents for liquid formulations and solid fillers for compositions in bars.

Preferably, the fragrance-modified branched polyorganosiloxanes according to the invention can be contained in laundry softener formulations. Apart from the fragrance-modified branched polyorganosiloxanes according to the invention, which also have a softening effect, they may also contain other laundry softening components that impart softness and antistatic properties to the treated fabrics. The laundry softening components can be selected from cationic, non-ionic, amphoteric or anionic laundry softening components. The quaternary ammonium compounds or their amine precursors are typical for the cationic softening components. Examples for laundry softening agents include: N,N-di(tallowyl-oxy-ethyl)-N,N-dimethyl ammonium chloride; N,N-di(canolyl-oxy-ethyl)-N,N-dimethyl ammonium chloride; N, N-di(tallowyl-oxy-ethyl)-N-methyl, N-(2-hydroxyethyl) ammonium chloride; N,N-di(canolyl-oxy-ethyl)-N-methyl,N-(2-hydroxyethyl) ammonium chloride; N,N-di(2-tallowyloxy-2-oxo-ethyl)-N,N-dimethyl ammonium chloride; N,N-di(2-canolyloxy-2-oxo-ethyl)-N,N-dimethyl ammonium chloride N,N-di(2-tallowyloxyethylcarbonyloxyethyl)-N,N-dimethyl ammonium chloride; N,N-di(2-canolyloxyethylcarbonyloxyethyl)-N,N-dimethyl ammonium chloride; N-(2-tallowoyloxy-2-ethyl)-N-(2-tallowyloxy-2-oxo-ethyl)-N,N-dimethyl ammonium chloride; N-(2-canolyloxy-2-ethyl)-N-(2-canolyloxy-2-oxo-ethyl)-N,N-dimethyl ammonium chloride; N,N,N-tri(tallowyl-oxy-ethyl)-N-methyl ammonium chloride; N,N,N-tricanolyl-oxy-ethyl)-N-methyl ammonium chloride; N-(2-tallowyloxy-2-oxoethyl)-N-(tallowyl)-N,N-dimethyl ammonium chloride; N-(2-canolyloxy-2-oxoethyl)-N-(canolyl)-N,N-dimethyl ammonium chloride; 1,2-ditallowyloxy-3-N,N,N-trimethylammoniopropane chloride; and 1,2-dicanolyloxy-3-N,N,N-trimethylammoniopropane chloride; and mixtures of the aforementioned agents.

Other examples for quaternary ammonium softening compounds include methyl bis(tallow amidoethyl)(2-hydroxyethyl)ammonium methyl sulfate and methyl bis(hydrated tallow amidoethyl)(2-hydroxyethyl)ammonium methyl sulfate; these materials are available from Momentive Performance Materials under the trade names Varisoft 222 and Varisoft 110. N,N-di(tallowyl-oxy-ethyl)-N,N-dimethyl ammonium chloride, in which the tallow chains are at least partially unsaturated, is particularly preferred. Other suitable examples for laundry softening agents are derived from fatty acyl groups in which the names “tallowoyl” and “canoloyl” in the above examples are replaced with the names “cocoyl, palmoyl, lauroyl, oleoyl, ricinoleoyl, stearoyl, palmitoyl”, which corresponds to the triglyceride source from which the fatty acyl units are derived. These alternative fatty acyl sources can comprise either fully saturated or preferably at least partially unsaturated chains. In the case of ester laundry softeners, the pH value of the compositions is of importance. The pH value is preferably in the range from 2.0 to 5, preferably in the range from 2.5 to 4.5, preferably from 2.5 to 3.5. In this case, the pH of these compositions can be adjusted by adding a Brönsted acid. Examples for suitable acids include inorganic mineral acids, carboxylic acids, in particular the low-molecular (C₁— to C₅—) carboxylic acids, and alkylsulfonic acids. Suitable inorganic acids include HCl, H₂SO₄, HNO₃ and H₃PO₄. Suitable organic acids include formic, acetic, citric, methylsulfonic and ethylsulfonic acids. Citric, hydrochloric, phosphoric, formic, methylsulfonic and benzoic acids are preferred acids. Other suitable quaternary ammonium laundry softener compounds are cationic nitrogen salts with two or more long-chain acyclic aliphatic C₈ to C₂₂-hydrocarbon groups or one of those groups and an aryl alkyl group, which can be used either alone or as a part of a mixture, and which are selected from the group consisting of (i) acyclic quaternary ammonium salts, (ii) diamino-alkoxylated quaternary ammonium salts and mixtures thereof. Examples include dialkyl dimethyl ammonium salts, such as ditallow dimethyl ammonium chloride, ditallow dimethyl ammonium methylsulfate, di(hydrated tallow)dimethyl ammonium chloride, distearyl dimethyl ammonium chloride, dibehenyl dimethyl ammonium chloride. Di(hydrated tallow)dimethyl ammonium chloride and ditallow dimethyl ammonium chloride.

Suitable amine laundry softener compounds are selected from (i) reaction products of higher fatty acids with a polyamine, selected from hydroxyalkylalkylenediamines and dialkylenetriamines and mixtures thereof. (With regard to further examples, see “Cationic Surface Active Agents as Fabric Softeners,” R. R. Egan, Journal of the American Oil Chemicals' Society, January 1978, pages 118-121).

Additional laundry softening materials can be used additionally or alternatively to the cationic laundry softener. They can be selected from non-ionic, amphoteric or anionic laundry softening materials. A disclosure of such materials can be found in U.S. Pat. No. 4,327,133; U.S. Pat. No. 4,421,792; U.S. Pat. No. 4,426,299; U.S. Pat. No. 4,460,485; U.S. Pat. No. 3,644,203; U.S. Pat. No. 4,661,269; U.S. Pat. No. 4,439,335; U.S. Pat. No. 3,861,870; U.S. Pat. No. 4,308,151; U.S. Pat. No. 3,886,075; U.S. Pat. No. 4,233,164; U.S. Pat. No. 4,401,578; U.S. Pat. No. 3,974,076; U.S. Pat. No. 4,237,016 and EP 472,178. Fatty acid partial esters of polyols or their anhydrides are preferred non-ionic softeners. Sorbitan esters and the glycerol esters are preferred non-ionic softeners.

Other laundry softener components that are suitable for use in this case are softener clay such as those with a low ion-exchange capacity as described in EP-A-0,150,531.

The laundry softener compounds are present in amount of 1% to 80% of the softener compositions or the laundry detergent composition.

Moreover, the softener compositions and/or laundry detergent compositions can contain, for example, brightening agents in amounts of 0.005% by wt. to 5% by wt., such as 4,4′-bis[(4-anilino-6-(N-2-bishydroxyethyl-)-s-triazine-2-yl)amino]-2,2′-stilbenedisulfonic acid and disodium salt thereof.

Moreover, the softener compositions and/or laundry detergent compositions can contain dispersing agents such as (1) cationic alkyl surfactants with long single chains; (2) non-ionic surfactants; (3) amine oxides; (4) fatty acids and (5) mixtures thereof in amounts of, for example, 2% by wt. to 25% by wt. of the composition. Examples for (1) are quaternary cationic monoalkylammonium compounds, such as quaternary ammonium salts of the general formula: [R_{long}N⁺(Rₛₕₒᵣₜ)₃]X⁻ wherein R_{long} is a C₈— to C₂₂-alkyl- or alkenyl group and Rₛₕₒᵣₜ is a C₁— to C₆-alkyl- or substituted alkyl group (e.g. hydroxyalkyl), a benzyl group, hydrogen, a polyethoxylated chain having 2 to 20 oxyethylene units and X⁻ is an anion. Examples include monolauryl trimethyl ammonium chloride and monotallow trimethyl ammonium chloride, monooleyl- or monocanola trimethyl ammonium chloride, monococonutoil trimethyl ammonium chloride, monosoybean trimethyl ammonium chloride.

Examples of (2) are non-ionic surfactants serving as viscosity/dispersability enhancers, and include addition products of ethylene oxide and optionally propylene exide, with fatty alcohols, fatty acids, fatty amines etc. In this case, the non-ionic surfactants are characterized by an HLB value (hydrophilic-lipophilic balance) of 7 to 20, preferably of 8 to 15.

Examples of (3) include amine oxides with an alkyl or hydroxyalkyl unit of 8 to 22 carbon atoms, such as dimethyloctylamineoxide, diethyldecylamineoxide, bis(2-hydroxyethyl)dodecylamineoxide, dimethyldodecylamineoxide, dipropyltetradecylamineoxide, methylethylhexadecylamineoxide, dimethyl-2-hydro-xyoctadecylamineoxide and coconut fat alkyldimethylamineoxide.

Laundry detergent or softening formulations containing fragrance-modified branched polyorganosiloxanes can additionally contain stabilizers such as antioxidants and reductants in a concentration of 0 to 2% by wt., such as a mixture of ascorbic acid, ascorbyl palmitate, propyl gallate, a mixture of BHT (butylated hydroxytoluene), BHA (butylated hydroxyanisol), propyl gallate and citric acid.

Moreover, laundry detergent or softening formulations containing fragrance-modified branched polyorganosiloxanes can contain dirt repellent agents or agents for washing out dirt in concentrations of 0% by wt. to 10% by wt.

Moreover, laundry detergent or softening formulations containing fragrance-modified branched polyorganosiloxanes can contain foam-inhibiting or sludge dispersing agents in an amount of for example more than 2%, preferably at least 4% relative to the formulation.

Moreover, laundry detergent or softening formulations containing fragrance-modified branched polyorganosiloxanes can contain bactericides, such as glutaraldehyde, formaldehyde, 2-bromo-2-nitropropane-1,3-diol, in amounts of for example 1 to 1,000 ppm by wt. of active substance.

Moreover, laundry detergent or softening formulations containing fragrance-modified branched polyorganosiloxanes can contain additional non-bonded perfumes. They can also be the fragrances used for producing the fragrance-modified branched polyorganosiloxanes according to the invention. They can be added in a concentration of for example 0 to 10% by wt.

Moreover, laundry detergent or softening formulations containing fragrance-modified branched polyorganosiloxanes can contain masking agents in a concentration of for example 0.1 to 15% by wt.

Moreover, laundry detergent or softening formulations containing fragrance-modified branched polyorganosiloxanes can contain crystal growth inhibiting components (inter alia limestone inhibition) in a concentration of for example 0.01 to 5% by wt., such as organophosphonic acid.

Moreover, laundry detergent or softening formulations containing fragrance-modified branched polyorganosiloxanes can contain enzymes, such as lipases, proteases, cellulases, amylases and peroxidases in amounts of for example 0.001 to 5% by wt.

Moreover, laundry detergent or softening formulations containing fragrance-modified branched polyorganosiloxanes can contain liquid carriers, such as water in amounts of more than 50% by wt. of the composition, or organic solvents, such as lower monoalcohols, such a esthanol, propanol, isopropanol or butanol, diols (glycol, etc.), triols (glycerol, etc.) and higher polyols.

Moreover, laundry detergent or softening formulations containing fragrance-modified branched polyorganosiloxanes can contain common textile treatment agents, such as for example colorants; preservatives; surfactants; anti-shrinkage agents; fabric crisping agents; spotting agents; germicides; fungicides; anti-oxidants; anti-corrosion agents, enzyme stabilizers, materials effective for inhibiting the transfer of dyes from one fabric to another during the cleaning process (i.e., dye transfer inhibiting agents), hydrotropes, processing aids, dyes or pigments, and the like.

The fragrance-modified branched polyorganosiloxanes of the invention can also be used in surface treatment agents in the broadest sense, where they attach to the surface because of their high substantivity, exuding fragrance over a long period of time. Typical examples for materials with surfaces to be treated include, for example, dishware, floors, bathrooms, toilets, carpets, kitchens, leather, car seats, litter or animal litter.

Because of their special properties, the polyorganosiloxanes according to the invention are capable of permanently attaching to the substrates treated with them, to modify the surface properties of the substrates in a beneficial way in the process and to simultaneously fixate the fragrance on the substrate, from where it is released over a long period of time.

Accordingly, the invention furthermore relates to detergents, care products, surface treatment agents and cosmetic agents containing at least one fragrance-modified polyorganosiloxane of the invention.

Moreover, the invention relates to a method for coating a substrate comprising the application of at least one polyorganosiloxane according to the invention onto the surface of the substrate.

Moreover, the invention relates to the use of fragrance-modified organosilanes for producing linear or branched fragrance-modified polyorganosiloxanes of the invention.

The invention is illustrated by the following example.

### EXAMPLE 1

Preparation of a condensation product from alpha-damascone with an alkoxy amino polysiloxane.

0.5 mole of the alkoxy amino polysiloxane NH₂CH₂CH₂CH₂—Si(OEt)₂—[Si(OMe)₂]₁₀—OSi(OEt)₂—CH₂CH₂CH₂NH₂ is added to 1 mole alpha-damascone and heated for 12 h at 125° C. under agitation. Volatile products are removed via a distillation bridge. Possible remaining volatile components are subsequently removed by distillation in a water-jet vacuum (18 mbar, 3h, 100-140° C.). The result is a dark amber oily liquid.

### EXAMPLE 2

Preparation of a condensation product from alpha-damascone with an alkoxy amino polysiloxane with further addition of phenylethanol.

0.5 mole of the alkoxy amino polysiloxane from Example 1 is added to 1 mole alpha-damascone and 0.1 mole phenylethanol and heated for 12 h to 140° C. under agitation. Volatile products such as ethanol are removed via a distillation bridge. Remaining volatile components are subsequently removed by distillation in a water-jet vacuum at 140° C. and 18 mbar over 2 hours. The result is a red-brown oily liquid.

### EXAMPLE 3

Preparation of a cross-linked condensation product from the reaction of alpha-damascone with an alkoxy amino siloxane.

In order to demonstrate that the product from example 1 comprises reactive (in this case, condesation-capable) groups, it was mixed in air with 50% air humidity with dibutyl tindilaurate at 25° C. under strong agitation. After 6 hours, the result is a dark amber viscous mass with a gelatinous consistency.

1. A fragrance-modified polyorganosiloxane comprising reactive groups.

2. The polyorganosiloxane of claim 1, wherein at least one reactive group is (a) capable of reacting with a substrate, (b) capable of condensation, or (c) both capable of reacting with a substrate and capable of condensation.

3. The polyorganosiloxane of claim 2, wherein the substrate is selected from the group consisting of: fibers, textiles, plasters, paper, wallpapers, molded articles, ceramics, paint layers, foils, hair, skin, wood, glass, and plastics.

4. The polyorganosiloxane of claim 2, wherein the reactive group is at least a group of the formula (I):
—SiR²ₙX₃₋ₙ(I), wherein n=0 to 2,
R² is selected from optionally substituted alkyl and phenyl,
X is selected from halogen, —OR³, —OC(O)R³, —N═CR³₂, —NR³₂, —NC(O)R³, —R⁴—Y,
wherein R³ is selected from the group consisting of hydrogen, optionally substituted alkyl, optionally substituted alkenyl, optionally substituted cycloalkyl and aryl,
R⁴ is a divalent optionally substituted organic residue optionally comprising one or more heteroatoms, and
Y is selected from the group consisting of —NCO, —OH, —NR³₂, —C(O)Cl, —SO₂Cl, —SO₂-vinyl, triazinyl, pyrimidinyl.

5. The polyorganosiloxane of claim 2, wherein the substrate has on the surface functional groups that are selected from the group consisting of: hydroxy, amino, carboxy, carbonyloxy (ester), disulfide, and mercapto.

6. The polyorganosiloxane of claim 2, obtainable (a) by reacting a functionalized polyorganosiloxane with a fragrance, or (b) by condensation of fragrance-modified polyorganosiloxanes or organosilanes.

7. The polyorganosiloxane of claim 6, wherein the fragrance comprises at least one functional group selected from the group consisting of keto, aldehyde and hydroxy groups.

8. The polyorganosiloxane of claim 6, wherein the functionalized polyorganosiloxane comprises at least one functional group capable of reacting with keto-, aldehyde- and/or hydroxy-functionalized fragrances.

9. The polyorganosiloxane of claim 6, wherein the functionalized polyorganosiloxane or organosilane comprise at least at least one functional group selected from the group consisting of aminoalkyl groups bonded to a silicon atom, hydroxyalkyl groups bonded to a silicon atom and alkoxy groups bonded to a silicon atom.

10. The polyorganosiloxane of claim 9, wherein the functional polyorganosiloxane or functional organosilane further comprises at least one primary or secondary amine residue.

11. A method of coating a substrate comprising applying at least one of claim 1 onto the surface of the substrate.

12. A method of producing a scent comprising reacting the polyorganosiloxane of claim 1 to release the fragrance.

13. A method of producing a scent comprising (a) treating a substrate with at least one polyorganosiloxane of claim 1, and (b) releasing the fragrance from the fragrance-modified substrate.

14. A method of producing the polyorganosiloxane of claim 1, comprising reacting functionalized polyorganosiloxanes with fragrances.

15. A method of producing the polyorganosiloxane of claim 1, comprising condensing fragrance-modified polyorganosiloxane or organosilanes.

16. The method of claim 14, further comprising applying the polysiloxane obtained therefrom to a carrier material.

17. The method of claim 16, wherein applying to a carrier material comprises spray drying or fluid bed granulation.

18. A process of making for producing linear or branched fragrance-modified polyorganosiloxanes comprising condensing fragrance-modified organosilanes.

19. A process for producing a fragrance-modified detergent, a fragrance-modified care product, a fragrance-modified surface treatment agent or a fragrance-modified cosmetic, comprising reacting the polyorganosiloxane of claim 1 with, respectively, a detergent, a care product, a surface treatment agent or a cosmetic.

20. A a fragrance-modified detergent, a fragrance-modified care product, a fragrance-modified surface treatment agent or a fragrance-modified cosmetic containing at least one polyorganosiloxane according to claim 1.

21. The fragrance-modified polyorganosiloxane of claim 1, further comprising at least one surface-active compound selected from anionic surfactants, cationic surfactants and non-ionic surfactants.

22. A non-aqueous composition comprising the fragrance-modified polyorganosiloxane of claim 1 and at least one organic medium.
